(19) United States
(12) Patent Application Publication
(10) Pub. No.: US 2021/0321944 A1
(43) Pub. Date: Oct. 21, 2021

(54) CATHETER TUBE UNIT USED FOR ELECTRODE CATHETER, METHOD FOR MANUFACTURING THE SAME, CATHETER TUBE, AND ELECTRODE CATHETER

(71) Applicant: J-SOL MEDICAL CO., LTD., Tokyo (JP)

(72) Inventor: Takeshi OOI, Tokyo (JP)

(73) Assignee: J-SOL MEDICAL CO., LTD., Tokyo (JP)

(21) Appl. No.: 17/265,682

(22) PCT Filed: Aug. 6, 2019
(86) PCT No.: PCT/JP2019/030929

§ 371 Date: Feb. 3, 2021

(30) Foreign Application Priority Data
Aug. 6, 2018 (JP) 2018-147587

(51) Int. Cl. A61B 5/00; A61B 5/25

(52) U.S. Cl. CPC A61B 5/6852; A61M 25/005; A61B 5/25

(57) ABSTRACT

A catheter tube unit 2a of the present invention is housed in a catheter tube 2 of an electrode catheter 1, and used for the catheter tube whose distal end is made bendable and stretchable through a wire 5 fixed to the distal end, when the wire is manipulated from a base end side. The catheter tube unit includes the wire, a long and integrated structure 10 including at least a core tube 6 through which the wire is inserted, a plurality of lead wires 7, 7 . . . arranged around an outer circumference of the core tube and connected to electrodes 4, 4 . . . , and a sheath 8 that envelopes the plurality of lead wires overall at least over the exterior, and an exterior tube 11 composed of an extrusion-molded article arranged over the outer circumferential face of the structure, while placing the structure as a core.

### TECHNICAL FIELD

The present invention relates to a catheter tube unit used for an electrode catheter, a method for manufacturing the same, a catheter tube, and an electrode catheter.

### BACKGROUND ART

An electrode catheter used for electrophysiological inspection aimed at determining cause of arrhythmia (see Patent Literature 1, etc.), also referred to as EP catheter, has been used in the field of arrhythmia treatment. Most of such EP catheter are foreign-made. Referring now to FIG. 10 for explaining the structure, a catheter tube 200 has a pre-formed exterior tube 111 made of resin; a core tube 106 inserted in the exterior tube 111 so as to allow a wire 105 to guide therethrough; and a plurality of lead wires 107, 107 . . . which are connected to inspection electrodes provided on the distal end side of the catheter tube 200, and are inserted in an internal space 201 formed between the inner circumferential face of the exterior tube 111 and the outer circumferential face of the core tube 106.

### CITATION LIST

### Patent Literature

Patent Literature 1: JP 2006-061350 A

### SUMMARY OF INVENTION

### Technical Problem

The structured catheter tube 200 has a large diameter, which typically measures 6 Fr (2 mm), 5 Fr (1.65 mm), 4 Fr (1.32 mm) and so forth. Meanwhile, thinning has been desired in view of reducing the number of points of venipuncture during inspection, shortening the operation time, and relieving load of hemostasis and so forth after the usage. Thinning down for example to approximately 1 mm and insertion of a deflectable mechanism that employs the wires 105 will, however, make it difficult to leave a sufficient internal space 201, so that the plurality of lead wires 107, 107 . . . cannot be inserted therein, thus making it unable to manufacture a thin electrode catheter.

The present invention was arrived at in consideration of the aforementioned situation, and an object thereof is to provide a catheter tube unit used for an electrode catheter which can be thinned even equipped with a deflectable mechanism, a method for manufacturing the same, a catheter tube, and an electrode catheter.

### Solution to Problem

Aimed at solving the problem, a catheter tube unit of the present invention is the one that is housed in a catheter tube of an electrode catheter, and used for the catheter tube whose distal end is made bendable and stretchable through a wire fixed to the distal end, when the wire is manipulated from a base end side, the catheter tube unit including:

the wire;

a long and integrated structure that includes at least a core tube through which the wire is inserted, a plurality of lead wires arranged around an outer circumference of the core tube and connected to the electrode, and a sheath that envelopes the plurality of lead wires overall at least over the exterior; and

an exterior tube composed of an extrusion-molded article arranged over the outer circumferential face of the structure, while placing the structure as a core.

A catheter tube of the present invention includes the aforementioned catheter tube unit, and an electrode unit coupled to the distal end of the catheter tube unit,

the electrode unit including an electrode tube, and a member housed in the electrode tube and capable of making the distal end of the catheter tube bendable and stretchable, and

the wire and the lead wires, extended from the catheter tube unit, being inserted in the electrode tube, the wire being fixed to the distal end side of the electrode tube, and the lead wires being connected to the electrode.

An electrode catheter of the present invention includes the aforementioned catheter tube, and a hand-piece arranged on a base end side of the catheter tube, and is capable of manipulating the wire so as to bend and stretch the distal end of the catheter tube.

A method for manufacturing a catheter tube unit of the present invention is the one for manufacturing a catheter tube unit that is housed in a catheter tube of an electrode catheter, and used for the catheter tube whose distal end is made bendable and stretchable through a wire fixed to the distal end, when the wire is manipulated from a base end side, the method including:

a step including arranging a plurality of lead wires connected to the electrode, on the outer circumference of the core tube in which the wire is inserted, and enveloping the plurality of lead wires overall at least over the exterior with a sheath, so as to obtain a long and integrated structure having at least the core tube, the plurality of lead wires and the sheath; and

a step of forming an exterior tube by extrusion molding, over an outer circumferential face of the structure placed as a core.

### Advantageous Effects of Invention

According to the present invention, the exterior tube is formed by extrusion molding while being integrated with the structure to be housed therein as a core, thus making it no longer necessary to insert the lead wire after the exterior tube is formed, and making it possible to arrange the lead wires without leaving the internal space between the exterior tube and the core tube. Hence, the electrode catheter may be thinned even equipped with the deflectable mechanism, enabling manufacture of the electrode catheter with a small diameter that could not be achieved by the previous product.

### BRIEF DESCRIPTION OF DRAWINGS

FIG. 1 is a side elevation schematically illustrating an embodiment of a catheter tube unit, a catheter tube, and an electrode catheter of the present invention.

FIG. 2 is a cross-sectional view of the catheter tube illustrated in FIG. 1, taken along line A-A′.

FIG. 3 is a vertical cross-sectional view of the catheter tube illustrated in FIG. 1, viewed from the viewer's side along line B-B′.

FIG. 4 is a vertical cross-sectional view taken perpendicularly to the longitudinal direction, schematically illustrating an exemplary method for manufacturing the catheter tube unit of the present invention.

FIG. 5 is a vertical cross-sectional view taken perpendicularly to the longitudinal direction, schematically illustrating an exemplary method for manufacturing the catheter tube unit of the present invention.

FIG. 6 is a vertical cross-sectional view taken perpendicularly to the longitudinal direction, schematically illustrating an exemplary method for manufacturing the catheter tube unit of the present invention.

FIG. 7 is a cross-sectional view taken in the direction of extrusion, when the exterior tube is extrusion-molded around the outer circumferential face of the structure placed as a core.

FIG. 8 is a cross-sectional view similar to FIG. 2, schematically illustrating another embodiment of the catheter tube unit of the present invention.

FIG. 9 is a cross-sectional view similar to FIG. 2, schematically illustrating still another embodiment of the catheter tube unit of the present invention.

FIG. 10 is a vertical cross-sectional view taken perpendicularly to the longitudinal direction, schematically illustrating a prior catheter tube used for an electrode catheter.

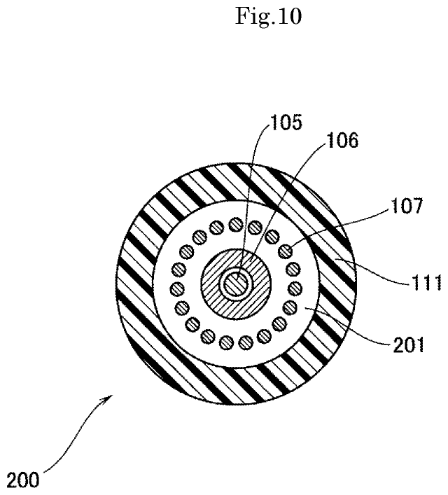

### DESCRIPTION OF EMBODIMENTS

Embodiments of the present invention will be explained below, referring to the attached drawings.

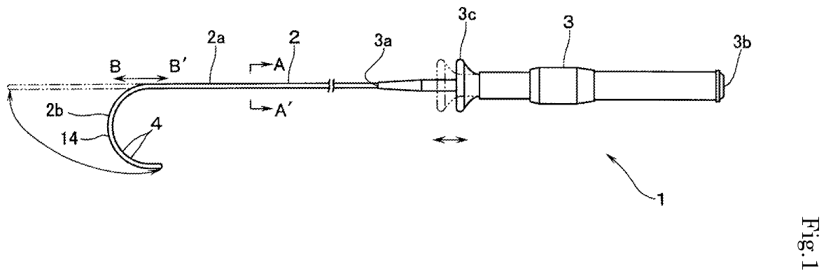

FIG. 1 illustrates a catheter tube unit 2a, catheter tube 2, and an electrode catheter 1 of this embodiment. The electrode catheter 1 is used for electrophysiological inspection aimed at determining cause of arrhythmia, and electrodes 4, 4 . . . of the catheter tube 2 are inserted into a living body, for example into a cardiac cavity and brought into contact with myocardium so as to measure action potential.

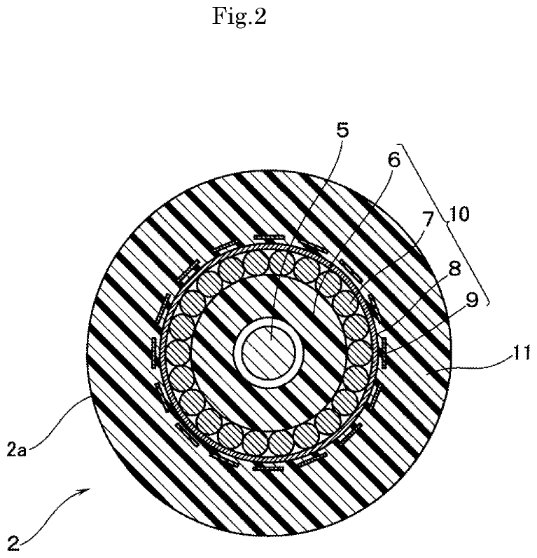

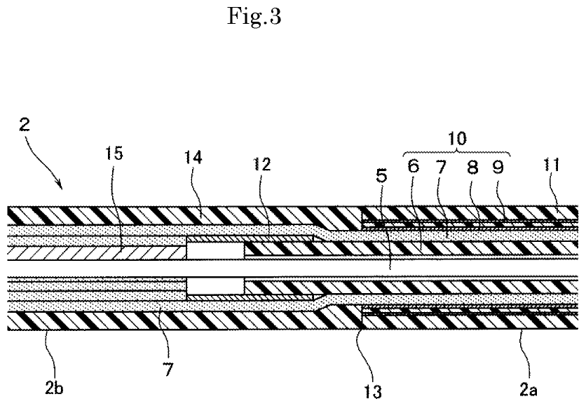

The catheter tube unit 2a of this embodiment builds up the catheter tube 2, together with an electrode unit 2b coupled to the distal end of the catheter tube unit 2a. The electrode catheter 1 has the catheter tube 2 and a hand-piece 3 attached to the base end side thereof. The catheter tube 2 has, as illustrated in FIGS. 2 and 3, a wire 5 housed therein. The wire 5 is fixed to the distal end of the catheter tube 2, so that the distal end of the catheter tube 2 can bend and stretch as illustrated in FIG. 1, when the wire is manipulated with the wire 5 by using the hand-piece 3 provided on the base end side of the catheter tube 2.

The catheter tube unit 2a has, as illustrated in FIGS. 2 and 3, the wire 5, a structure 10, and an exterior tube 11. The structure 10 has a core tube 6, a plurality of lead wires 7, 7 . . . , a sheath 8, and a braid 9.

The wire 5 is inserted in the core tube 6, so as to freely movable in the longitudinal direction. The wire 5 may have any cross-sectional shape not specifically limited, but preferably has a nearly circular shape. The wire 5 preferably has a diameter of 0.3 mm or smaller, which is more preferably 0.2 mm or smaller, although not specifically limited.

Material for composing the wire 5 is exemplified by, but not limited to, metals such as stainless steel and nickel-titanium-based alloy. Alternatively, it is not always necessarily composed of a metal, instead allowing use of high-strength nonconductive wire. The wire 5 may be either single wire or stranded wire.

The core tube 6 allows the wire 5 to be inserted therein, and guides it in the longitudinal direction. The core tube 6 may have any shape of pipe, coil and so forth, so long as it can form a tube, and preferably has a pipe form. The core tube 6 may have any vertical cross-sectional shape not specifically limited, but preferably has a nearly circular shape. The core tube 6 may have an outer diameter not specifically limited so long as the wire 5 can be inserted therein, and a desired number of lead wires 7, 7 . . . may be arranged on the outer circumferential face, which is preferably 0.2 to 0.5 mm from the viewpoint of thinning, and more preferably 0.3 to 0.4 mm. The core tube 6 has an inner diameter slightly larger than the outer diameter of the wire 5.

Material for composing the core tube 6 is exemplified by, but not specifically limited to, organic polymer material, and metal materials such as stainless steel. Among them, the organic polymer material, featured by its high insulating property, is suitable as a material for composing the electrode catheter 1. Above all, low friction material is preferred from the viewpoint of reducing sliding resistance between the core tube 6 and the wire 5 inserted therein, wherein the material is exemplified by fluorine-containing resins such as polytetrafluoroethylene and ethylene-tetrafluoroethylene copolymer, polyether ether ketone, nylon 66, high-density polyethylene, and polyamide 12. The core tube 6, when made of a resin material, is obtainable by extrusion molding.

The plurality of lead wires 7, 7 . . . are principally connected to the electrodes 4, 4 . . . of the catheter tube 2, and are arranged around the outer circumference of the core tube 6. Each lead wire 7 extends in the longitudinal direction in contact with the core tube 6. The plurality of lead wires 7, 7 . . . are arranged typically as illustrated in FIG. 2, but not limited thereto, wherein every adjacent lead wires 7, 7 are preferably kept in close contact, and each of the plurality of lead wires 7, 7 . . . are arranged in contact with the outer circumferential face of the core tube 6. With such an arrangement, a large number of lead wires 7 to be connected to the plurality of electrodes 4, 4 . . . may be housed even when the catheter tube unit 2a has a small diameter.

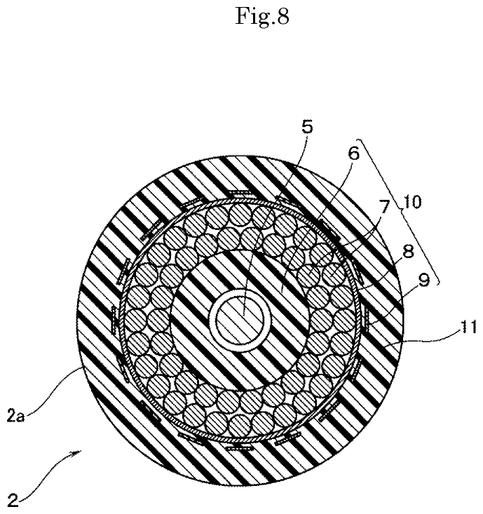

Alternatively, depending on occasion and requirement, at least a part of adjacent lead wires 7, 7 may be arranged while being kept apart; the plurality of lead wires 7, 7 . . . may be arranged while being helically twisted on the outer circumferential face of the core tube 6; or as illustrated in FIG. 8, another plurality of lead wires 7, 7 . . . may further be arranged on the outer circumferential face of the plurality of lead wires 7, 7 . . . having been arranged in contact with the outer circumferential face of the core tube 6 to form a multi-stage arrangement.

Each lead wire 7 preferably has an insulating sheath arranged around a core wire. An employable lead wire 7 typically has the core wire made of low-resistivity gold, silver, copper or the like, and formed therearound the insulating sheath made of polyurethane, enamel, fluorine-containing resin or the like. Diameter of the lead wire 7, including the core wire and the insulating sheath, is not specifically limited but preferably 0.05 to 0.12 mm, not only from the viewpoint of thinning, but also from sharpness of display of intracardiac potential, avoidance of drift in intracardiac potential, and avoidance of risk of wire breakage, which is more preferably 0.05 to 0.08 mm. The number of the plurality of lead wires 7, 7 . . . is preferably 10 or larger, in view of obtaining a plurality of types of information on intracardiac potential, which is more preferably 20 or larger.

The sheath 8 covers at least the exterior of the plurality of lead wires 7, 7 . . . overall, to protect the plurality of lead wires 7, 7. The sheath 8 in this embodiment is arranged on the outer circumferential face of the plurality of lead wires 7, 7 . . . overall. The sheath 8 typically prevents dielectric breakdown of the lead wire 7 due to heat during extrusion molding of the exterior tube 11, and prevents breakage of the lead wire 7 in the process of manufacturing the catheter tube unit 2a. Material for composing the sheath 8 is not specifically limited, instead allowing use of various insulating materials such as organic polymer material.

The sheath 8 may be arranged over the outer circumferential face of the plurality of lead wires 7, 7 . . . overall, typically by wrapping the sheath 8 around the outer circumferential face of the plurality of lead wires 7, 7 . . . overall.

The sheath 8 may be arranged over the outer circumferential face of the plurality of lead wires 7, 7 . . . overall, alternatively by enveloping them overall with a heat-shrinkable tube whose diameter will shrink under heating, and then by externally applying heat typically by allowing them to pass through a heating oven, to thereby shrink the heat-shrinkable tube.

Again alternatively, the sheath 8 may be arranged over the outer circumferential face of the plurality of lead wires 7, 7 . . . overall, by inserting the core tube 6 with the plurality of lead wires 7, 7 . . . arranged on the outer circumferential face thereof, into a resin tube having an equivalent inner diameter.

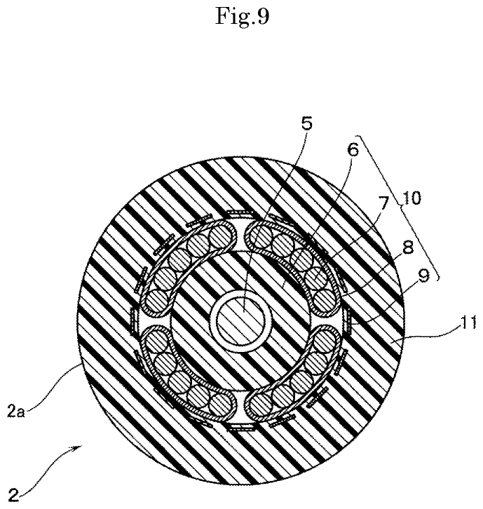

Referring now to an embodiment illustrated in FIG. 9, also in a case of using lead wire groups each having the sheath 8 that covers all around the lead wires 7, 7 . . . , the sheath 8 can be provided to cover all around the lead wires 7, 7 . . . by choosing an appropriate one of the aforementioned methods.

The braid 9 is arranged on the outer circumference of the sheath 8. The braid 9 is embedded in the exterior tube 11 during extrusion molding of the exterior tube 11 with use of the structure 10 placed as the core, functions as a reinforcing member of the catheter tube 2, can provide torque transmission property that enables transmission of rotational force applied on the base end of the electrode catheter 1 to the distal end, and can provide kink resistance sufficient for preventing kink from occurring within a meandering blood vessel. The braid 9 may be arranged by any method without special limitation, exemplified by winding. The braid 9 is formed by crossing or weaving of metal strands. The braid 9 employable here may be any of those based on known technologies which can be referred to in the field of catheter tube. Material composing the metal strands for the braid 9 is exemplified by, but not limited to, stainless steel, tungsten, tantalum, nickel-titanium-based alloy, cobalt-chromium-based alloy and amorphous alloy. The braid 9 may alternatively be made of a material other than metal, that is, resin for example. The braid 9 composed of resin is exemplified by a tube made of a resin having the melting point higher than that of the exterior tube 11, wherein such resin is exemplified by polyimide, polyamide, polyether ether ketone, and liquid crystal polymer.

The aforementioned core tube 6, the plurality of lead wires 7, 7 . . . , the sheath 8 and the braid 9 together build up the long and integrated structure 10, while arranging the lead wire 7, 7 . . . , the sheath 8 and the braid 9 on the outer circumferential face of the core tube 6 placed at the center.

The exterior tube 11 is an extrusion-molded article arranged over the outer circumferential face of the structure 10, while placing the structure 10 as a core. According to the catheter tube unit 2a of the present embodiment, the exterior tube 11 is formed by extrusion molding while being integrated with the structure 10 to be housed therein as a core, thus making it no longer necessary to insert the lead wire 7 after the exterior tube 11 is formed, and making it possible to arrange the lead wires 7 without leaving the internal space between the exterior tube 11 and the core tube 6. Hence, the electrode catheter 1 may be thinned even equipped with the deflectable mechanism, enabling manufacture of the electrode catheter 1 with a small diameter that could not be achieved by the previous product. This makes it possible to reduce the number of points of venipuncture during inspection, shorten the operation time, and relieve load of hemostasis after the usage.

Material for composing the exterior tube 11 employable here may be, but not specifically limited to, any of thermoplastic molding materials applicable to extrusion molding, and is exemplified by various flexible resins that include polyolefins such as polypropylene and ethylene-vinyl acetate copolymer; polyamide; polyesters such as polyethylene terephthalate (PET) and polybutylene terephthalate (PBT); polyurethane; polyvinyl chloride; polystyrene-based resin; fluorine-containing resins such as polytetrafluoroethylene and ethylene-tetrafluoroethylene copolymer; polyamide elastomer such as polyether block amide copolymer; polyurethane elastomer; polystyrene elastomer; polyester elastomer; fluorine-containing elastomer; rubber materials such as silicone rubber and latex rubber; and any combined materials of two or more materials enumerated above.

The structured catheter tube unit 2a of this embodiment may have the outer diameter, which is not specifically limited but preferably 1.32 mm or smaller from the viewpoint of thinning, and is more preferably 1 mm or smaller.

Note that, although the structure 10 of the catheter tube unit 2a illustrated in FIGS. 1 to 3 is composed of the core tube 6, the plurality of lead wires 7, 7 . . . , the sheath 8 and the braid 9, the structure 10 may alternatively be built without using the braid 9.

That is, the catheter tube unit 2a is featured by having i) the wire 5; ii) the long and integrated structure 10 that includes at least the core tube 6 through which the wire 5 is inserted, the plurality of lead wires 7, 7 . . . arranged around the outer circumference of the core tube 6 and connected to the electrodes 4, 4 . . . , and the sheath 8 that envelopes the plurality of lead wires 7, 7 . . . overall at least over the exterior; and iii) the exterior tube 11 composed of an extrusion-molded article arranged over the outer circumferential face of the structure 10, while placing the structure 10 as a core. The structure 10 may have the braid 9 arranged on the outer circumference of the sheath 8.

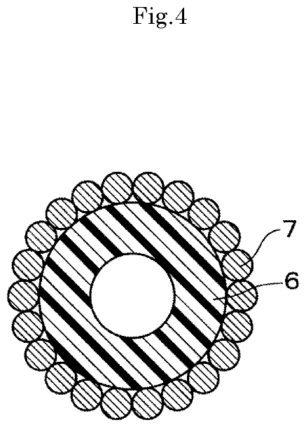

The catheter tube unit 2a may be manufactured by a method that includes the steps below, as illustrated in FIGS. 4 to 7:

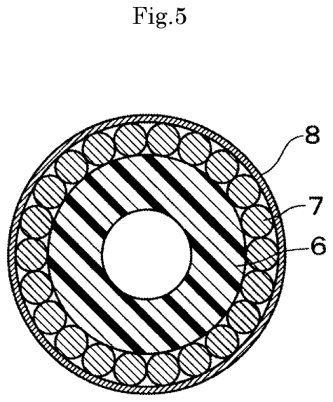

a step that includes (A) arranging the plurality of lead wires 7, 7 . . . connected to the electrodes 4, 4 . . . , on the outer circumference of the core tube 6 in which the wire 5 is inserted (FIG. 4); and (B) enveloping the plurality of lead wires 7, 7 . . . overall at least over the exterior with the sheath 8 (FIG. 5), so as to obtain the long and integrated structure 10 having at least the core tube 6, the plurality of lead wires 7, 7 . . . and the sheath 8; and

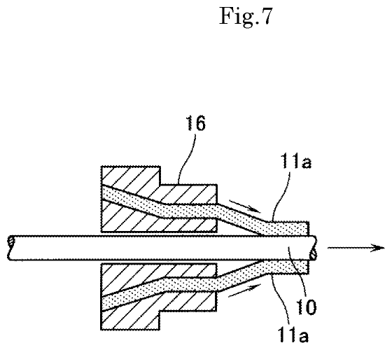

a step of forming an exterior tube 11 by extrusion molding, over the outer circumferential face of the structure 10 placed as a core (FIG. 7).

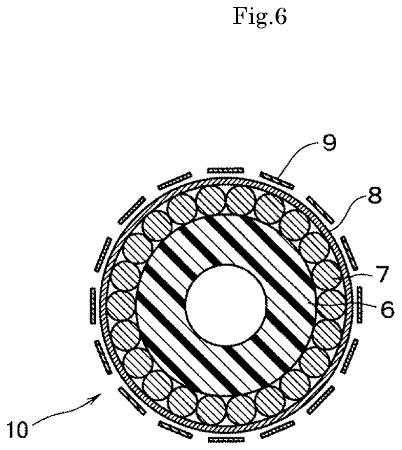

The structure 10 may further have the braid 9 arranged around the outer circumference of the sheath 8, as illustrated in FIG. 6.

Having described this embodiment referring to FIGS. 4 to 7 that illustrate the procedure in which step (A) comes first and step (B) comes next, the procedure allows free choice without being limited thereto, exemplifying an embodiment illustrated in FIG. 9, in which the lead wire groups are preliminarily manufactured by enveloping the lead wires 7, 7 . . . overall with the sheath 8, and then arranging them around the outer circumferential face of the core tube 6.

In the method for manufacturing the catheter tube unit 2a, the exterior tube 11 is extrusion-molded, as illustrated in FIG. 7, typically by using a ring die 16 having an extrusion path formed therein, inserting the structure 10 into a cavity of the ring die 4, extruding a thermoplastic molding material 11a through the extrusion path of the ring die 4 in a tube shape onto the outer circumferential face of the structure 10, while moving the structure 10 in one direction, thereby covering the entire outer circumferential face of the structure 10 with the molding material 11a. In this way, the exterior tube 11 may be formed by using the molding material 11a on the outer circumferential face of the structure 10.

The catheter tube 2 that uses the aforementioned catheter tube unit 2a of this embodiment has, as illustrated in FIGS. 1 and 3, the catheter tube unit 2a, and an electrode unit 2b coupled to the distal end of the catheter tube unit 2a.

The electrode unit 2b has an electrode tube 14 having arranged therein the electrodes 4, 4 . . . for inspection, and a leaf spring 15 illustrated in FIG. 3, which is a component housed in the electrode tube 14, and is capable of making the distal end of the catheter tube 2 bendable and stretchable.

As seen in FIG. 3, the distal end of the catheter tube unit 2a and the base end of the electrode tube 2b are coupled by a coupling part 13. Mode of coupling of the catheter tube unit 2a and the electrode tube 2b is not specifically limited, so long as they are prevented from separating.

From the distal end of the catheter tube unit 2a, extend are the wire 5 and the plurality of lead wires 7, 7 . . . . That is, the exterior tube 11, the sheath 8 and so forth are peeled off at the distal end of the catheter tube unit 2a manufactured by the aforementioned method, and also the core tube 6 is cut off while leaving a part thereof that extends from the distal end of the catheter tube unit 2a, to thereby allow the wire 5 and the plurality of lead wires 7, 7 . . . hidden therein to expose. To the electrode tube 2b, inserted are wire 5 and the plurality of lead wires 7, 7 . . . that are extended from the catheter tube unit 2a.

A resin sleeve 12 is attached to the core tube 6 protruded out from the distal end of the catheter tube unit 2a by fitting. Although the electrode tube 2b might be made of a tube equally flexible with the catheter tube unit 2a, the electrode tube 2b which resides at the distal end of the catheter tube 2 is made relatively flexible, meanwhile the catheter tube unit 2a which resides on the base end side is made relatively rigid. The thickness of the exterior tube 11 may be same or different between the electrode tube 2b and the catheter tube unit 2a. In the example illustrated in FIG. 3, the electrode tube 2b is made so that the outer circumference is matched to the outer diameter of the exterior tube 11, meanwhile made thinner than the exterior tube 11 of the catheter tube unit 2a, in order to achieve flexibility enough to make the distal end of the catheter tube 2 bendable and stretchable. Hence, there remains a clearance between the inner circumferential face of the electrode tube 2b and the core tube 6, even with the plurality of lead wires 7, 7 . . . laid in between. The electrode tube 2b is therefore, as illustrated in FIG. 3, given the thickness equivalent to the exterior tube 11 partially at the base end, and a sleeve 12 having a larger diameter than the core tube 6 is fitted to the coupling part 13 while holding the plurality of lead wires 7, 7 . . . therebetween, thus coupling the distal end of the catheter tube unit 2a and the base end of the electrode tube 2b.

Material for composing the electrode tube 2b is exemplified by, but not limited to, the molding materials and so forth enumerated previously as the materials for composing the exterior tube 11.

The wire 5 inserted into the electrode tube 2b is fixed to the distal end of the electrode tube 2b. The wire 5 is arranged on one face of the leaf spring 15 inside the electrode tube 2b. Mode of fixation of the wire 5 to the distal end of the electrode tube 2b is not specifically limited, instead allowing fixation to the distal end of the leaf spring 15, or to a component or the like attached to the distal end of the electrode tube 2b, typically by way of soldering, laser welding, ultrasonic welding, arc welding or brazing.

Then, upon pulling of the wire 5 at the base end of the catheter tube 2, the leaf spring 15 warps, and the distal end of the catheter tube 2 is bent. Upon relaxing the wire 5 from the tensed state, the distal end of the bent catheter tube 2 can be stretched to recover the original shape with the aid of elastic resilience of the leaf spring 15. Such a mechanism per se, capable of bending and stretching the distal end of catheter through manipulation on the base end side, has been already known and put into practical use, typically as described in Patent Literature 1 and JP 05-507212 A.

Material for composing the leaf spring 15 is exemplified by, but not specifically limited to, stainless steel, nickel-titanium alloy, cobalt-nickel alloy, and polymer materials such as fluorine-containing resin and polyamide resin.

A component housed in the electrode tube 14 and capable of bending or stretching the distal end of the catheter tube 2 is not specifically limited so long as it enables the wire 5 to operate in this way, and even may be a component other than the leaf spring 15. A known mechanism typically has ring components coupled with a wire or the like.

As seen in FIG. 3, the plurality of lead wires 7, 7 . . . that extend from the distal end of the catheter tube unit 2a are connected to the electrodes 4, 4. Of the plurality of lead wires 7, 7 . . . , a part of them may otherwise be drawn out from the distal end of the catheter tube unit 2a, depending on the number of the electrodes 4, 4 . . . .

Mode of arrangement, layout, the number of arrangement and so forth of the electrodes 4, 4 . . . may be determined referring to known technologies in the field of electrode catheter tube, for which any structure based on such known technologies is applicable. The electrodes 4, 4 . . . , although schematically exemplified in FIG. 1, allow free choice of arrangement, layout, the number of arrangement and so forth depending on the purposes and so forth. For example, annular electrodes 4, 4 . . . having the diameter equivalent to the outer diameter of the catheter tube 2 may be arranged at regular intervals or different intervals in the longitudinal direction, a hole through which the lead wire 7 can pass may be bored in the electrode tube 14, and a core wire of the lead wire 7 may be once drawn out through the hole to the outside, and then connected to the electrode 4. The electrodes 4, 4 . . . may be arranged at regular intervals; two electrodes 4, 4 may be paired and then arranged at regular intervals by twos; or the electrode 4 may be arranged at the distal end of the electrode tube 14. The number of electrodes 4 is preferably 1 to 40 and more preferably 2 to 24, although the maximum number of accommodation of which is limited by the outer diameter of the catheter tube 2. Alternatively, a part of the inspection electrodes of the electrode catheter 1 may reside in the catheter tube unit 2a, wherein a part of the plurality of lead wires 7, 7 . . . in the catheter tube unit 2a is connected to the electrodes.

Material for composing the electrode 4 is exemplified by, but not specifically limited to, aluminum, copper, stainless steel, gold, platinum, iridium, rhenium and alloy.

Length of the catheter tube 2 beyond the hand-piece 3 is preferably, but not limited to, 60 to 180 cm for the convenience of measurement of cardiac action potential.

The catheter tube 2, making use of the aforementioned catheter tube unit 2a of this embodiment is used as the electrode catheter 1 as illustrated in FIG. 1. The electrode catheter 1 has the catheter tube 2, and the hand-piece 3 arranged on the base end side of the catheter tube 2, and is capable of manipulating the wire 5 so as to bend and stretch the distal end of the catheter tube 2.

The hand-piece 3 is structured as a cashing which can be gripped by the user, and has a mechanism for manipulating the wire 5. The catheter tube 2 is inserted from a distal end 3a of the hand-piece into the casing. In the casing, the wire 5 and the plurality of lead wires 7, 7 . . . extend from the base end of the catheter tube 2. The base end of the wire 5 is fixed to the inside of the casing of the hand-piece 3.

The mechanisms for manipulating the wire 5 in the hand-piece 3 may be determined referring to known technologies in the field of electrode catheter tube, without special limitation, for which any structure based on such known technologies is applicable. Such known technologies typically include a mechanism by which a component movable in the axial direction or a pivotable component, which are connected to the wire 5, is manipulated by moving or rotating it in or around the axial direction; and a mechanism by which movement of the wire 5 in the axial direction is converted into pivoting movement by using a screw mechanism, so as to manipulate a pivoting component. In FIG. 1, there is provided a movable component 3c that is connected to the wire 5 and can move in the axial direction. When the movable component 3c is pinched and pulled towards the operator's side, the wire 5 is tensed to make the leaf spring 15 warp at the distal end of the catheter tube 2, to thereby bend the distal end of the catheter tube 2. Upon pushing the movable component 3c forward in the axial direction, the wire 5 relaxes from the tensed state, and the bent distal end of the catheter tube 2 stretches to recover the original shape with the aid of elastic resilience of the leaf spring 15.

At a hand-piece rear end 3b of the hand-piece 3, there is provided a connector to which the plurality of lead wires 7, 7 . . . are drawn out from the base end of the catheter tube 2.

FIG. 8 is a cross-sectional view similar to FIG. 2, schematically illustrating another embodiment of the catheter tube unit of the present invention. In this embodiment, the plurality of lead wires 7, 7 . . . arranged around the outer circumference of the core tube 6 and connected to the electrode 4, 4 . . . are stacked in the thickness direction to form two layers. The sheath 8 entirely envelopes the inner lead wires 7, 7 . . . arranged all around the core tube 6 in contact with the outer circumferential face thereof, and the outer lead wires 7, 7 . . . arranged all around the inner lead wires 7, 7 . . . , so that the sheath 8 is brought into contact with the outer lead wires 7, 7 . . . , thus making the sheath 8 envelope the plurality of lead wires 7, 7 . . . overall at least over the exterior.

FIG. 9 is a cross-sectional view similar to FIG. 2, schematically illustrating still another embodiment of the catheter tube unit of the present invention. In this embodiment, the plurality of lead wires 7, 7 . . . connected to the electrodes 4, 4 . . . are divided into a plurality of bundles, and a lead wire group of each bundle is independently enveloped by the separate sheath 8. Thus the sheaths 8, 8 . . . envelope the plurality of lead wires 7, 7 . . . overall at least over the exterior.

According to the manufacturing method applicable to this case, the structure 10 is obtainable by enveloping each lead wire group all around with the sheath 8, and then by arranging the lead wire groups thus enveloped by the sheaths 8 around the outer circumferential face of the core tube 6.

While FIG. 9 illustrates an exemplary case having a plurality of (four) lead wire groups, the structure 10 according to another example of the present invention may have all lead wires 7, 7 . . . preliminarily enveloped by a single sheath 8, and may have such single lead wire group, enveloped by the sheath 8, arranged around the outer circumference of the core tube 6. That is, acceptable is an embodiment having arranged therein only one lead wire group, unlike the plurality of lead wire groups as illustrated in FIG. 9.

While FIG. 9 illustrates an exemplary case having the lead wires 7, 7 . . . arranged to form a single layer in the thickness direction within each lead wire group, the lead wires 7, 7 . . . may alternatively be arranged to form two layers within each lead wire group in the thickness direction, as illustrated in FIG. 8.

Having descried the embodiments of the present invention, the present invention is by no means limited to these embodiments, instead allowing various modifications without departing from the spirit thereof. For example, while this embodiment has described referring to the electrode catheter 1 (EP catheter) used for electrophysiological inspection aimed at determining cause of arrhythmia, the present invention is also applicable to an abrasion catheter used for a therapeutic method by which the catheter is inserted deep into a myocardial tissue that causes arrhythmia in heart, and high-frequency current is then applied from the electrode to the myocardial tissue or the periphery, so as to cauterize them (abrasion) and to induce coagulation necrosis, thus breaking an arrhythmic circuit; and also applicable to any inspection and measurement of electric signals of human body, including electroencephalography and myocardial potential test. The electrode catheter in the present invention is intended to be applied to any part of the body, without limiting purposes of use.

### REFERENCE SIGNS LIST

1 Electrode catheter
2 Catheter tube
2a Catheter tube unit
2b Electrode unit
3 Hand-piece
3a Distal end of a hand-piece
3b Rear end of a hand-piece
3c Movable component
4 Electrode
5 Wire
6 Core tube
7 Lead wire
8 Sheath
9 Braid
10 Structure
11 Exterior tube
11a Molding material
12 Sleeve
13 Coupling part
14 Electrode tube
15 Leaf spring
16 Ring die
105 Wire
106 Core tube
107 Lead wire
111 Exterior tube
200 Catheter tube
201 Internal space

1. A catheter tube unit that is housed in a catheter tube of an electrode catheter, and used for the catheter tube whose distal end is made bendable and stretchable through a wire fixed to the distal end, when the wire is manipulated from a base end side, the catheter tube unit comprising:
the wire;
a long and integrated structure including at least a core tube through which the wire is inserted, a plurality of lead wires arranged around an outer circumference of the core tube and connected to the electrode, and a sheath that envelopes the plurality of lead wires overall at least over the exterior; and
an exterior tube composed of an extrusion-molded article arranged over the outer circumferential face of the structure, while placing the structure as a core.

2. The catheter tube unit according to claim 1, wherein the structure has a braid arranged on an outer circumference of the sheath.

3. A catheter tube comprising the catheter tube unit described in claim 1, and an electrode unit coupled to a distal end of the catheter tube unit,
the electrode unit including an electrode tube, and a member housed in the electrode tube and capable of making the distal end of the catheter tube bendable and stretchable, and
the wire and the lead wires, extended from the catheter tube unit, being inserted in the electrode tube, the wire being fixed to the distal end side of the electrode tube, and the lead wires being connected to the electrode.

4. An electrode catheter comprising the catheter tube described in claim 3, and a hand-piece arranged on a base end side of the catheter tube, and is capable of manipulating the wire so as to bend and stretch the distal end of the catheter tube.

5. A method for manufacturing a catheter tube unit that is housed in a catheter tube of an electrode catheter, and used for the catheter tube whose distal end is made bendable and stretchable through a wire fixed to the distal end, when the wire is manipulated from a base end side, the method comprising:
a step including arranging a plurality of lead wires connected to the electrode, on the outer circumference of the core tube in which the wire is inserted, and enveloping the plurality of lead wires overall at least over the exterior with a sheath, so as to obtain a long and integrated structure having at least the core tube, the plurality of lead wires and the sheath; and
a step of forming an exterior tube by extrusion molding, over an outer circumferential face of the structure placed as a core.

6. The method for manufacturing a catheter tube unit according to claim 5, the method further comprising arranging a braid on an outer circumference of the sheath, so as to obtain the long and integrated structure having at least the core tube, the plurality of lead wires, the sheath and the braid.

7. A catheter tube comprising the catheter tube unit described in claim 2, and an electrode unit coupled to a distal end of the catheter tube unit,
the electrode unit including an electrode tube, and a member housed in the electrode tube and capable of making the distal end of the catheter tube bendable and stretchable, and
the wire and the lead wires, extended from the catheter tube unit, being inserted in the electrode tube, the wire being fixed to the distal end side of the electrode tube, and the lead wires being connected to the electrode.

8. An electrode catheter comprising the catheter tube described in claim 7, and a hand-piece arranged on a base end side of the catheter tube, and is capable of manipulating the wire so as to bend and stretch the distal end of the catheter tube.
